(19) United States
(12) Patent Application Publication
(10) Pub. No.: US 2012/0142811 A1
(43) Pub. Date: Jun. 7, 2012

(54) BONE CEMENT COMPOSITION

(76) Inventors: Richard Kowalski, Preston (GB); Diane Baker, Ellesmere (GB)

(21) Appl. No.: 13/032,078

(22) Filed: Feb. 22, 2011

Related U.S. Application Data
(62) Division of application No. 12/066,105, filed on Dec. 2, 2008, now abandoned.

(30) Foreign Application Priority Data
Jul. 8, 2005 (GB) 0514076.9

(51) Int. Cl. A61L 24/06; C08F 26/02; C08F 20/28; C08L 33/12; C08F 20/34; C08K 3/32; C08K 3/30; C08F 20/14; C08F 24/00

(52) U.S. Cl. 523/116; 526/325; 526/301; 526/320; 526/270; 526/312; 524/417; 524/423; 524/561

(57) ABSTRACT

A bone cement composition contains (a) the product of a polymerisation reaction between a high molecular weight dimethacrylate monomer having a molecular weight of at least 250 and bearing at least one hydrophilic group, a monofunctional methacrylate monomer having a molecular weight of not more than 250 bearing at least one hydrophilic group, an methacrylate monomer, and a polymer having a molecular weight of at least 200,000, and (b) an inorganic filler which is present in an amount of at least about 40% by weight, based on the total weight of the cement composition.

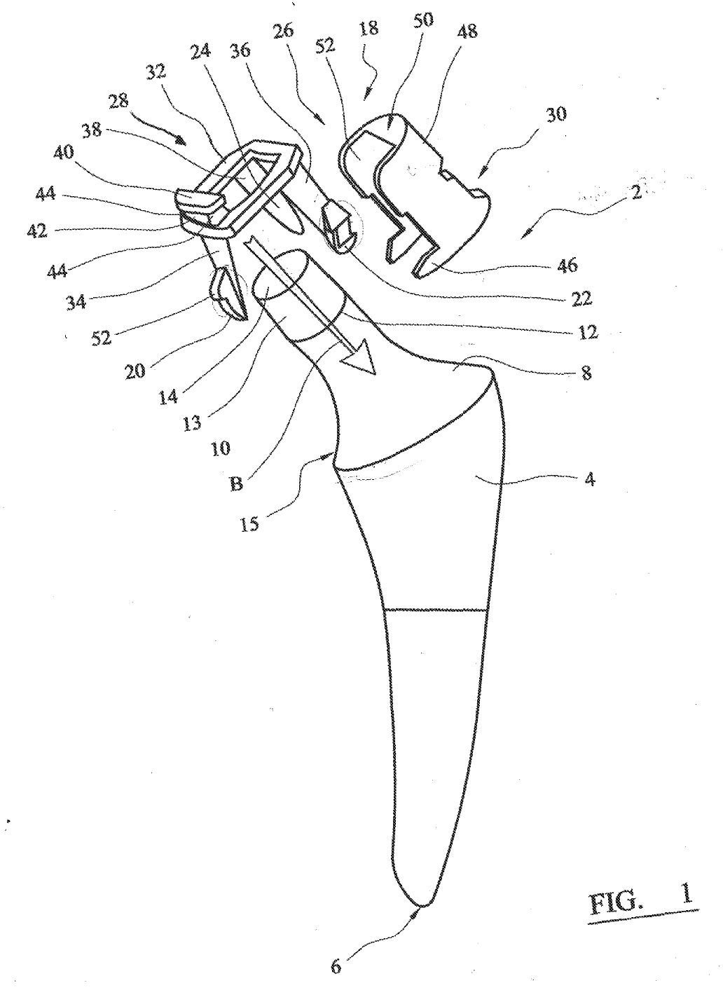

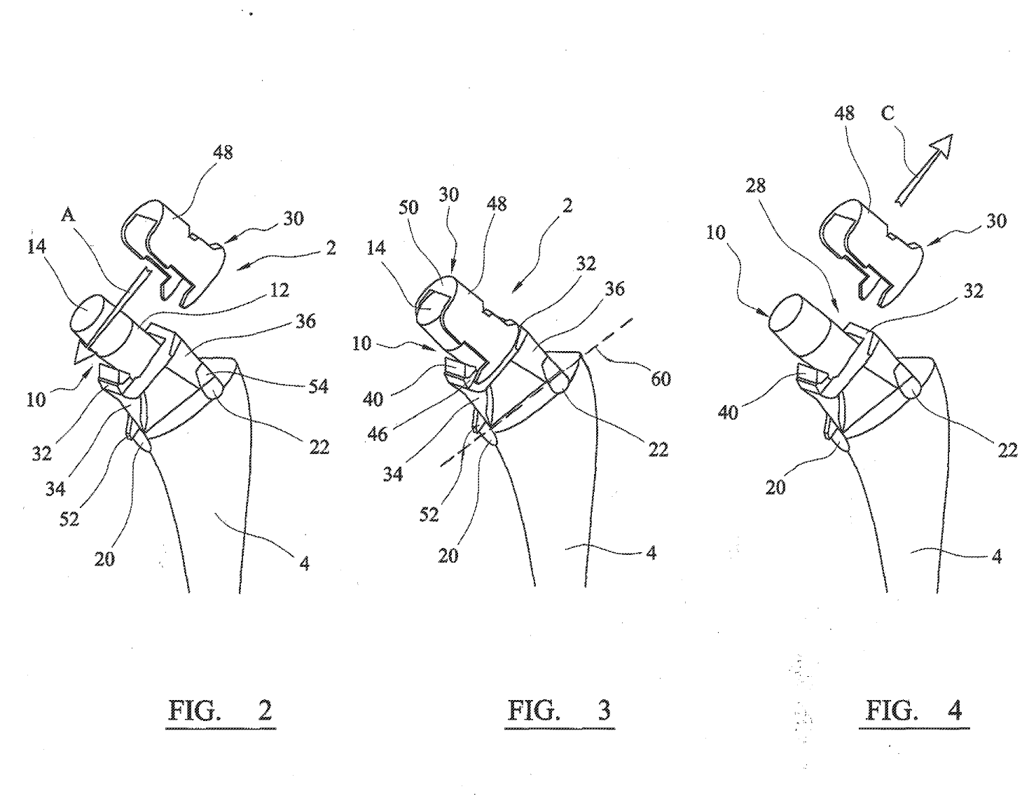

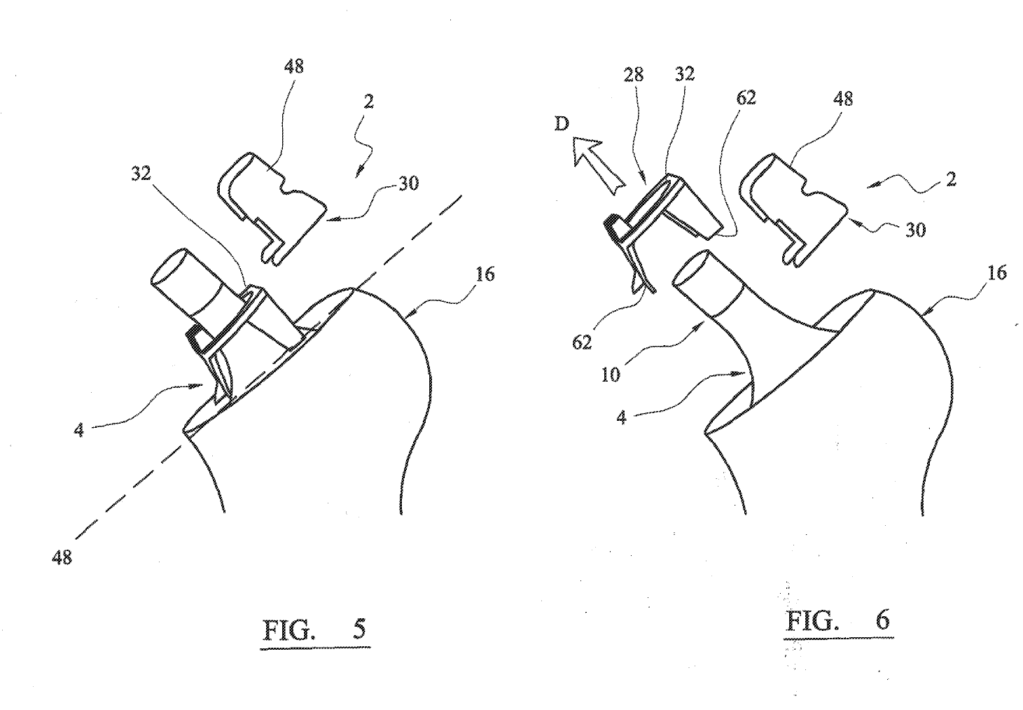

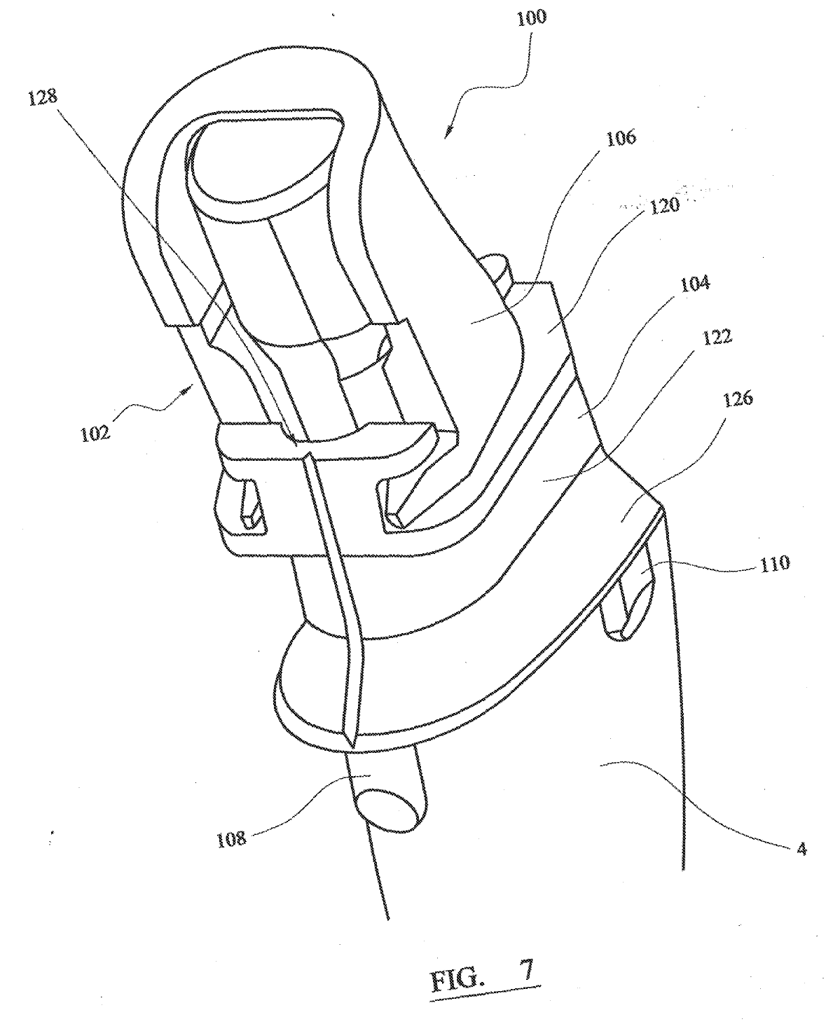

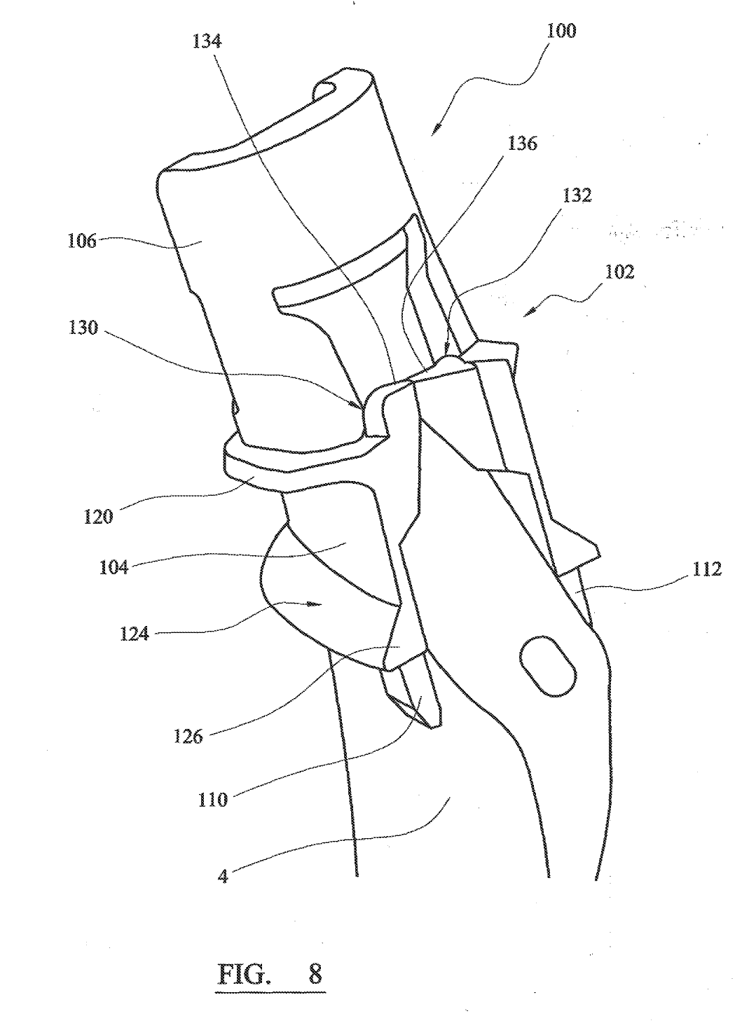

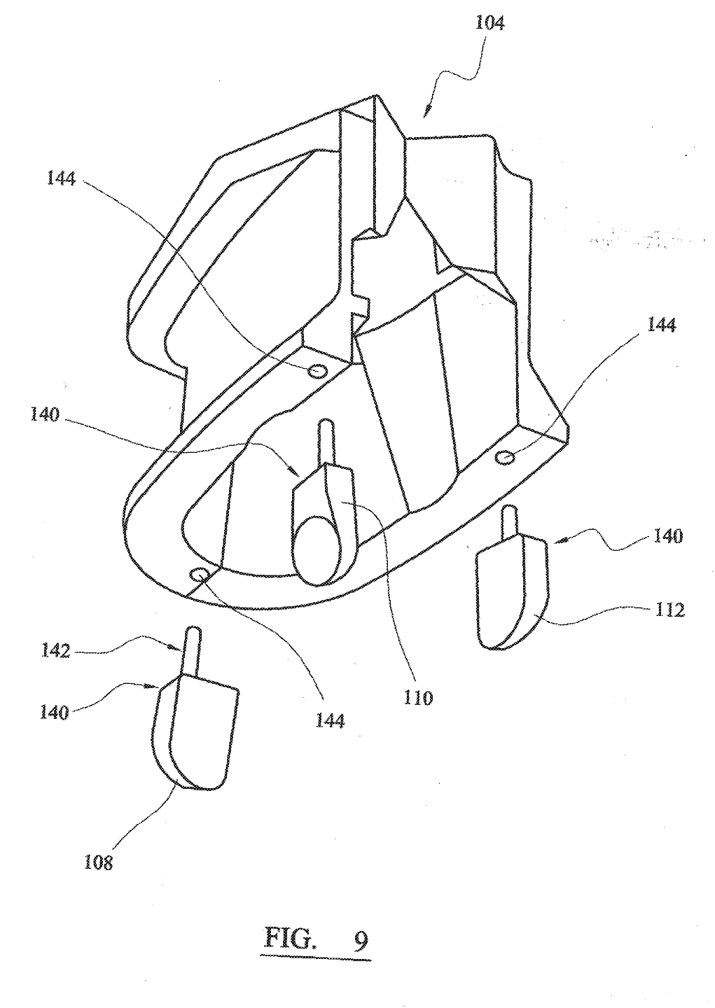

### BACKGROUND OF THE INVENTION

The present invention relates to formulations for bone cement compositions. Such compositions can be used in the treatment of bone defects or fractures arising both from procedures for treatment of conditions arising from natural degeneration and trauma, and in the fixation of prostheses and other implant articles.

The present invention therefore seeks to provide cement formulations for bonding to bone for use in surgical procedures.

A number of attempts have been carried out to provide bone cement which is mechanically stable in order to provide good fixation in orthopaedic applications, and which can stimulate a bone reaction which is consistent with strong fixation. Glass ionomer cements are used in dentistry but lack the mechanical strength for use in orthopaedic applications. Calcium phosphate cements can be seen sometimes as having the disadvantage that they are too weak to be used in weight-bearing indications without the use of pins or screws etc.

Cement formulations containing a high loading of tricalcium phosphate (TCP) in dimethacrylate monomer (bis-GMA) have been tried but suffer the disadvantage of high losses of tricalcium phosphate due to the hydrophilic nature of the monomer. The monomer causes the TCP to resorb or dissolve too quickly to allow good bone attachment. A further problem arises in that hydrophilic monomers such as Bis-GMA, urethane dimethacrylate monomer (UDMA) and tetrahydrofurfuryl methacrylate monomer (THFMA) can only be made into paste systems because they cannot attack sufficiently the polymer powder in order to form a dough. Consequently, cements made from these monomers do not have the desired handling characteristics for good cementation. It is also difficult to formulate a cement which has more that 50% (by dry weight) of inorganic material powder in the formulation because the handling characteristics are poor and the powder is very dry. Another problem arises in that it is difficult to sterilise paste systems. The present invention provides a bone cement system which includes a liquid component and a powder component, in which the liquid component is formulated so that the properties arising from the constitution of the powder component can be optimised.

### BRIEF SUMMARY OF THE INVENTION

Accordingly, in one aspect, the invention provides a liquid formulation for use in preparing bone cement comprising 5 to 35% by weight of a high molecular weight dimethacrylate monomer having a molecular weight of at least 250 and bearing at least one hydrophilic group, 5 to 35% by weight of a monofunctional methacrylate monomer having a molecular weight of not more than 250 bearing at least one hydrophilic group, 30 to 90% by weight of a methacrylate monomer. Preferably, the methacrylate monomer which makes up 30 to 90% by weight of the liquid formulation is methyl methacrylate. Preferably, the liquid formulation includes an activator for a polymerisation reaction between monomer molecules.

In another aspect, the present invention provides a powder formulation for use in preparing bone cement, the powder comprising an inorganic filler in an amount of from 20 to 85% by weight of the powder formulation, and one or more biocompatible polymers having a molecular weight of at least 200,000 in an amount of from 15 to 80% by weight of the powder formulation. Preferably, the powder formulation includes an initiator for a polymerisation reaction involving the biocompatible polymer. The initiator can be present in an amount of 0.10 to 2.0% by weight of the powder formulation.

In a further aspect, the invention provides a bone cement composition formed by mixing a liquid formulation as discussed above with a powder formulation as discussed above, in a ratio of from 1.9 to 2.9 parts by weight of powder to 1.0 part by weight of liquid.

In yet another aspect, the invention provides a bone cement composition which comprises (a) the product of a polymerisation reaction between a high molecular weight dimethacrylate monomer having a molecular weight of at least 250 and bearing at least one hydrophilic group, a monofunctional methacrylate monomer having a molecular weight of not more than 250 bearing at least one hydrophilic group, an methacrylate monomer, and a polymer having a molecular weight of at least 200,000, and (b) an inorganic filler which is present in an amount of at least about 40% by weight, based on the total weight of the cement composition.

### DETAILED DESCRIPTION OF THE INVENTION

Molecular weight values which are referred to in this specification are weight average molecular weights.

A bone cement can be produced from the liquid and powder formulations mentioned above by a copolymerisation reaction between the polymerisable components of the liquid and powder formulations. The formation of cements by reactions of this kind is well known. The reaction can involve initiation by appropriate activators or initiators or both, as is known. For example, the reaction can be initiated by an initiator which can initiate the polymerisation reaction on contact with the liquid formulation. Preferred initiators include organic peroxides such as benzoyl peroxide. The initiator can be provided in the powder formulation or can be provided separately. When the initiator is provided in the powder formulation, it is preferably present in an amount of at least about 0.10% by weight of the powder formulation, more preferably at least about 0.20%. Preferably, the initiator is present in an amount of not more than about 2.0% by weight of the powder formulation, more preferably not more than about 1.0%.

Initiation of the reaction can involve use of an activator. Examples of suitable activators include certain tertiary amines, including N,N-dimethyl-p-toluidine or N,N-dihydroxyethyl-p-toluidine. When the activator is a liquid, it can be convenient in some systems to provide this as a component of the liquid formulation.

The polymerization reaction can then be initiated when the initiator and activator come into contact with one another during the physical mixing of the powder and liquid.

It can be preferred for the liquid formulation also to include an inhibitor to inhibit polymerisation of the monomer components. This can help to reduce the spontaneous polymerisation reaction which can be observed with certain monomers such as methacrylate monomers under normal storage conditions. Suitable inhibitors include hydroquinone, monomethylhydroquinone and butylated hydroxytoluene. The inhibitor can be present in an amount of 0.001 to 1.00% by weight.

The high molecular weight polymer in the powder formulation should be capable of participating in the polymerisation of polymerisable components of the liquid formulation in order to form an interpenetrating network between the polymerisable components of the liquid formulation and the high molecular weight polymer in the powder formulation. Preferably, the high molecular weight polymer in the powder formulation or the long chain monomer in the liquid formulation, or more preferably each of them, should be capable of participating in a cross-linking reaction. Cross-linking can add structural integrity to the cured cement.

Preferably, the high molecular weight polymer in the powder formulation is soluble in the liquid formulation when the liquid and powder formulations are mixed, for example so as to form a solution or a gel. The characteristics of the mixture are also affected by the presence of inorganic filler. The formation of a gel by dissolution of the powder in the liquid can help to provide good handling characteristics, for example by conferring dough-like or pasty characteristics to the mixed components.

A preferred long chain monomer in the liquid formulation should contain hydrophilic groups. Preferred long chain monomers include urethane dimethacrylate (UDMA), bis-glycol dimethacrylate (bis-GMA), polyethylene glycol dimethacrylate (PEGDMA).

A preferred short chain monomer should contain hydrophilic groups. An example of a preferred monofunctional methacrylate monomer includes tetrahydrofurfuryl methacrylate (THFMA).

Preferably, the methacrylate-based monomer of the liquid formulation is methyl methacrylate. Examples of hydrophilic groups present on the long and short chain monomers include oxygen-containing groups and nitrogen-containing groups. Specific groups include oxygen containing heterocyclic groups, hydroxyl groups and amine or amide groups.

Preferably, the filler in the powder formulation preferably acts as a source of calcium ions. Preferably, the calcium ions are capable of being leached from the cement composition when the composition has been implanted in a patient. Release of calcium ions can help to a bone reaction which is consistent with strong fixation. For example, it can help to stimulate the growth of bone tissue, in particular into pores in the bone cement which result from leaching of the calcium ions from the cement material after the material has cured. The incorporation of a filler which has these properties renders the composition “bioactive”.

Preferred fillers include calcium phosphates (such as beta-tricalcium phosphate (β-TCP)), calcium sulphate, and bioglass compositions. It will generally be preferred that the calcium containing compound composition is chosen so that it resorbs slowly on the surface of the cement but does not resorb or dissolve out of the cement too quickly to prevent osteoconduction from occurring. It is also important that degradation of the cement does not occur to an unacceptable extent.

Preferably, the filler is present in the powder formulation in an amount of at least about 20%, more preferably at least about 30%, especially at least about 50%, by weight of the powder formulation. Preferably, the filler is present in the powder formulation in an amount of not more than about 90%, more preferably not more than about 85%, especially not more than about 75% by weight of the powder formulation.

Preferably, ratio by weight of the powder and liquid formulations which are mixed to form the cement is at least about 1.5, more preferably at least about 1.9, especially at least about 2.0, for example at least about 2.12. Preferably, the value of that ratio is not more than about 3.2, more preferably not more than about 2.9, especially not more than about 2.75, for example not more than about 2.57.

Preferably, the amount of the filler in the cement composition is at least about 10% by weight, based on the total weight of the composition, more preferably at least about 25%, especially at least about 40%, for example at least about 50% or at least about 51%. Preferably, the amount of the filler in the cement composition is not more than about 70% by weight, based on the total weight of the composition, more preferably not more than about 65%, especially not more than about 63%, for example not more than about 55%.

Preferably, the inorganic filler has a median particle size measured using a Beckman Coulter laser diffraction particle size analyser which is at least about 1 μm, more preferably at least about 3 μm. Preferably, the size of the inorganic filler particles is not more than about 20 μm, more preferably not more than about 15 μm.

The high molecular weight polymer in the powder may be a single polymer or a combination of polymers. It can help to modify the viscosity of the material of the anchor layer in the period before it hardens.

In this embodiment in order to achieve dough-like consistency viscous monomers are used together with the polymerisable components in the liquid formulation and a high molecular weight polymer in the powder formulation. Suitable polymers include poly(methyl methacrylate), copolymers of methyl methacrylate. Suitable copolymers are made from monomers by copolymerisation with methyl methacrylate and such monomers include styrene, ethyl methacrylate, butyl methacrylate and methyl acrylate. Methyl acrylate-methylmethacrylate copolymer is the preferred copolymer.

Preferably, the molecular weight (number average) of the high molecular weight polymer in the powder formulation is at least about 200,000. More preferably, its molecular weight is at least about 500,000, and most preferably is at least about 750,000. Preferably, the high molecular weight polymer is present in the powder formulation in an amount of at least about 15% by weight of the powder formulation, more preferably at least about 25%, especially at least about 40%. Preferably, the high molecular weight polymer is present in the powder formulation in an amount of not more than about 85% by weight of the powder formulation, more preferably not more than about 80%, especially not more than about 70%.

Preferably, the resulting bone cement has a compressive strength measured by the method of ISO 5833 of at least 70 MPa.

The use of a filler which can act as a source of calcium ions has the advantage of stimulating bone growth, at least under favourable conditions. Bone can grow into the cement composition over an extended period, for example of six months or more, to a depth of several micrometres, for example to a depth of at least about 50 μm, or at least about 100 μm, preferably at least about 250 μm, more preferably at least about 1 mm, and possibly to greater depths, for example at least about 2 mm, or at least about 4 mm.

The use of the combination of monomers in the liquid formulation, in conjunction with the polymer component in the powder formulation, can help to confer useful handling characteristics on the cement composition, notwithstanding the presence of a high loading of an inorganic filler material. Accordingly, the cement composition can have the consistency of a dough rather than a paste. This allows the handling characteristics of the cement of the present invention to be compatible with current surgical cementation techniques. This can be contrasted with known cement compositions with a high proportion of filler (for example, 50% by weight or more) which have tended to have paste-like consistencies.

The use of high inorganic filler loadings in the cement is facilitated by use of the monomer mixture used in the liquid component. The monomer mixture of the present invention ensures that the dispersion of the filler which is formed on mixing the powder and liquid is homogenous. A disadvantage of prior art compositions is that the dispersion of the filler formed on mixing may not be homogenous. Without wishing to be bound by theory, it is believed that the hydrophilic properties arising as a blend of the monomers used in the present invention assist in controlling the dispersion of the hydrated filler. It is also believed that the liquid monomer mixture can have an effect on the elution of calcium from the filler and also on the water uptake. The degree to which these effects are manifested may be controlled by varying the total monomer quantity in the cement composition and also the composition of the liquid.

The bone cement composition of the present invention addresses some or all of the problems of the prior art. In particular, known bone cements commonly comprise an inorganic powder filler and a curable liquid monomer. If the resulting cured cement is too hydrophilic then a water soluble filler such as calcium phosphate will be resorbed or dissolved too quickly for good long term fixation to bone tissue. The compositions of the present invention can help to minimise this problem.

The bone cement composition of the invention can be used in the treatment of bone defects, and in the treatment of fractures. It can be used in the treatment of conditions arising from natural degeneration and trauma. The composition of the invention finds particular application in the fixation of prostheses and other implant articles.

The properties of the cement composition of the invention can be used to advantage in other applications. For example, the ability of the composition to stimulate the growth of bone tissue, in particular into voids in the bone cement which result from leaching of the calcium ions from close to the surface of the cement material after the material has cured, means that the cement composition can be used, after it has been cured, as an implant material. The properties of the material can then facilitate fixation of the implant. For example, the composition of the invention can be used to make an implant which is to be placed in contact with a patient's bone to fill a defect in the patient's natural tissue. This might be a defect in a bone. The material can be used in an anchor layer of an implant, for example for use in repair to cartilage defect. A cartilage repair implant is disclosed in an international application which is being filed with the present application, claiming priority from UK patent application no. 0514074.4. Subject matter which is disclosed in the specification of that application is incorporated in the specification of this application by this reference.

The present invention will now be illustrated by the following examples.

### Example 1

A powder formulation was produced containing:

| Constituent | % w/w |
|---|---|
| β-TCP | 79.75 |
| Benzoyl peroxide | 0.25 |
| Methyl acrylate-methyl methacrylate copolymer | 20.00 |

A liquid formulation was produced containing:

| Constituent | % w/w |
|---|---|
| Methylmethacrylate monomer | 66.0 |
| Urethane dimethacrylate monomer | 16.5 |
| Tetrahydrofurfuryl methacrylate monomer | 16.5 |
| Dimethyl-p-toluidine | 1.0 |
| Hydroquinone | 50 ppm |

The powder formulation was mixed with the liquid formulation in the ratio 2.13 parts by weight powder to 1 part by weight liquid formulation.

### Example 2

A powder component was produced containing:

| Constituent | % w/w |
|---|---|
| β-TCP | 70.0 |
| Benzoyl peroxide | 6.60 |
| Methyl acrylate-methyl methacrylate copolymer | 29.40 |

A liquid formulation was produced containing:

| Constituent | % w/w |
|---|---|
| Methylmethacrylate monomer | 66.0 |
| Urethane dimethacrylate monomer | 16.5 |
| Tetrahydrofurfuryl methacrylate monomer | 16.5 |
| Dimethyl-p-toluidine | 1.0 |
| Hydroquinone | 50 ppm |

The powder formulation was mixed with the liquid formulation in the ratio 2.33 parts by weight powder to 1 part by weight liquid formulation.

### Example 3

A powder component was produced containing:

| Constituent | % w/w |
|---|---|
| β-TCP | 79.5 |
| 33% benzoyl peroxide in dicalcium phosphate | 0.8 |
| Methyl acrylate-methyl methacrylate copolymer | 19.7 |

A liquid formulation was produced containing:

| Constituent | % w/w |
|---|---|
| Methylmethacrylate monomer | 66.0 |
| Urethane dimethacrylate monomer | 16.5 |
| Tetrahydrofurfuryl methacrylate monomer | 16.5 |
| Dimethyl-p-toluidine | 1.0 |
| Hydroquinone | 50 ppm |

The powder formulation was mixed with the liquid formulation in the ratio 2.0 parts by weight powder to 1.0 part by weight liquid formulation.

### Results

The compositions of the present invention have been evaluated in vivo. Eight week old Wistar rats weighing 180 to 230 grams were used in an implantation study. The rats were operated on under general anaesthesia introduced by intraperitoneal injection of sodium-5-ethyl-5-(1-methylbutyl) barbiturate (known as Nembutal and obtainable from Dainippon Pharmaceutical Co, Osaka, Japan) at 40 mg/kg body weight. Cortical bone defects measuring 2 H 7 mm were introduced into each of the rats at the medial aspect of the proximal metaphysis of both tibiae, and bone marrow was curetted. The intramedullary canals of both bone defects were irrigated with physiological saline and a paste-form cement was inserted at random.

Each paste was allowed to cure in situ for evaluation of osteoconductivity. A total of 30 rats were evaluated, with one leg for the composition of Example 3 and one leg for the CMW1 composition. CMW1 is commercially available polymethyl methacrylate bone cement available from DePuy International Limited and was used as a control. Six rats were sacrificed respectively at 4, 8, 12, 26 and 52 weeks after the operation.

The powder of Example 3 was composed of methyl methacrylate-methyl acrylate copolymer, benzoyl peroxide in inert dicalcium phosphate base and β-TCP. The average particle size of the β-TCP was 8 Φm. The liquid of Example 3 was composed of methyl methacrylate monomer, urethane dimethacrylate monomer, tetrahydrofurfuryl methacrylate monomer and dimethyl-p-toluidine and hydroquinone. The compositions of the powder and liquid are shown above in Example 3. The weight ratio of powder to liquid was approximately 2.25. The β-TCP was present in an amount of over 50% (approximately 53%) by weight of the total weight of the cement in Example 3.

The cement formed from the powder and liquid formulations of Example 3 was able to contact the bone directly without intervening soft tissue in the specimens at each time interval. Contact micro radiography revealed that this cement contacted the bone directly. There was always a soft tissue layer between CMW1 and bone. It was also revealed that the invasion of bony tissue into marginal cement of Example 3 was observed within 4 weeks and deeper invasion was observed at longer time intervals.

Histological findings demonstrate that the cement of Example 3 was biocompatible and the bony tissue invasion into cement of Example 3 seen in scanning electron microscope photographs indicated no toxic effect of the cement component.

It was also demonstrated that this cement was an excellent osteoconductive material as over half of the cement margin contacted directly to bone within 4 weeks of implantation and a larger part did by 26 weeks after implantation. The cement of Example 3 thus demonstrated a clear advantage over the sample CMW1.

1. A liquid formulation for use in preparing bone cement comprising 5 to 35% by weight of a high molecular weight dimethacrylate monomer having a molecular weight of at least 250 and bearing at least one hydrophilic group, 5 to 35% by weight of a monofunctional methacrylate monomer having a molecular weight of not more than 250 bearing at least one hydrophilic group, 30 to 90% by weight of an methacrylate monomer.

2. The liquid formulation of claim 1, wherein the long-chained monomer is selected from the group comprising urethane dimethacrylate (UDMA), polyethylene glycol dimethacrylate (PEGDMA) and bisglycol dimethacrylate (bis-GMA).

3. The liquid formulation of claim 1, wherein the short chain monomer is a hydrophilic monomer which is not capable of cross-linking.

4. The liquid formulation of claim 1, in which the short chain monomer is tetrahydrofurfuryl methacrylate (THFMA).

5. The liquid formulation of claim 1, in which the methacrylate monomer which makes up 30 to 90% by weight of the liquid formulation is methyl methacrylate.

6. The liquid formulation of claim 1, in which the hydrophilic groups are oxygen-containing and/or nitrogen-containing groups.

7. The liquid formulation of claim 1, which includes an inhibitor in an amount of 0.001 to 1.00% by weight which inhibits polymerisation of polymerisable components of the formulation.

8. A powder formulation for use in preparing bone cement, the powder comprising an inorganic filler in an amount of from 20 to 85% by weight of the powder formulation, one or more biocompatible polymers having a molecular weight of at least 200,000 in an amount of from 15 to 80% by weight of the powder formulation, and an initiator in an amount of 0.10 to 2.0% by weight of the powder formulation.

9. The powder formulation of claim 8, in which the inorganic filler is a calcium containing compound.

10. The powder formulation of claim 9, in which the calcium containing compound is selected from the group comprising: calcium phosphate (e.g. beta-tricalcium phosphate), calcium sulphate and bioglass.

11. The powder formulation of claim 8, in which the initiator is an organic peroxide.

12. The powder formulation of claim 8, in which the high molecular weight polymer includes one or more biocompatible polymers having a molecular weight of at least 200,000 in an amount of from 15 to 80% by weight and an initiator in an amount of 0.10 to 2.0% by weight of the powder composition.

13. A bone cement composition formed by mixing a liquid formulation as claimed in claim 1 with a powder formulation as claimed in claim 8, in a ratio of from 1.9 to 2.9 parts by weight of powder to 1.0 part by weight of liquid.

14-15. (canceled)
